(19) United States
(12) Patent Application Publication
(10) Pub. No.: US 2025/0205171 A1
(43) Pub. Date: Jun. 26, 2025

(54) TRANSDERMAL DELIVERY SYSTEMS FOR CERTAIN FACTOR Xa INHIBITORS AND RELATED METHODS FOR THEIR PREPARATION AND USE

(71) Applicant: ARx, LLC, Glen Rock, PA (US)

(72) Inventors: Jilin Zhang, York, PA (US); Scott Barnhart, York, PA (US)

(73) Assignee: ARx, LLC, Glen Rock, PA (US)

(21) Appl. No.: 18/846,426

(22) PCT Filed: Mar. 13, 2023
(86) PCT No.: PCT/US2023/015080

§ 371 Date: Sep. 12, 2024

Related U.S. Application Data
(60) Provisional application No. 63/319,714, filed on Mar. 14, 2022.

(51) Int. Cl. A61K 9/70; A61K 31/4545; A61K 47/10; A61K 47/12

(52) U.S. Cl. CPC A61K 9/7069; A61K 9/7061; A61K 31/4545; A61K 47/10; A61K 47/12

(57) ABSTRACT

Transdermal delivery systems, such as patches. for certain direct factor Xa inhibitors (“FXa”), as well as methods for the manufacture and use of such transdermal patches. particularly in the treatment of thrombosis or in other conditions wherein an anticoagulant is desired. and to prevent stroke and embolism in people with atrial fibrillation (AF).

### FIELD OF THE INVENTION

The present invention relates to a patch for transdermal delivery of certain factor Xa inhibitors (“FXa”), as well as methods for the manufacture and use of such transdermal patches.

### BACKGROUND OF THE INVENTION

Transdermal patches are widely used as a means of administering an active pharmaceutical ingredient (API) to a patient in need of therapeutic treatment. Advantages of transdermal patches as a dosage form, relative to other dosage forms, include precise and constant delivery of an API which provides for a relatively constant concentration of the API in the blood, avoidance of first-pass effect, and increased compliance relative to oral dosage forms as a patient need not remember to take tablets or capsules at specific times. Transdermal patches also provide further advantages relative to topical formulations, as the latter may be wiped off the skin accidentally or may undesirably contaminate other areas of the skin or other individuals who contact the topical formulation.

Generally, transdermal patches comprise a backing layer that is impermeable to the API, and a layer of an API-containing adhesive composition. The adhesive in such compositions comprise at least one adhesive polymer that enables the API-containing adhesive composition to adhere onto a patient's skin. Adhesives commonly included in these compositions are pressure sensitive adhesives (PSAs) that are (meth) acrylate-, silicone-, or polyisobutylene-based adhesives.

The development of transdermal patches can be complex, with issues arising from, for example, intrinsic API aqueous solubility, API-excipient and excipient-excipient incompatibilities, which in some formulations requires the further addition of other excipients which may, in turn, result in additional incompatibilities and/or an undesirable increase in cost. In addition, and even if the foregoing incompatibilities are overcome, certain APIs are difficult to formulate in a manner that provides acceptable levels of API concentrations in the bloodstream over a desired period of time, this inability to provide adequate API concentrations being due in large measure to the barrier properties of human skin.

FXas are known, as a class, to be effective anticoagulants, and in many cases must be administered as part of a regimen of treating a chronic ailment, such as thrombosis. A subclass of FXas, which may be referred to as direct factor Xa inhibitors (xabans), are anticoagulants used to both treat and prevent blood clots in the circulatory system, (e.g., after knee and hip replacement surgery) and to prevent stroke and embolism in people with atrial fibrillation (AF). Direct factor Xa inhibitors block the enzyme called factor Xa, preventing the conversion of prothrombin to thrombin in the final common pathway of clot formation in veins and the heart. However, when (commonly) administered orally, some FXas, such as the direct FXa apixaban, may cause, in some patients, gastrointestinal tract bleeding.

It has been difficult to formulate direct FXas into therapeutically effective, and regulatory approvable, transdermal dosage forms, including, in particular, a subclass of direct FXas that includes betrixaban, edoxaban, fondaparinux, rivaroxaban, darexaban, otamixaban, letaxaban, eribaxaban and apixaban.

### DETAILED DESCRIPTION OF THE INVENTION

The present invention was developed to provide for the transdermal delivery of a one or more of direct FXas to patients in need, as it is difficult to deliver therapeutically effective amounts of these direct FXa into a patient's bloodstream via the transdermal route.

The present invention, provides, in one aspect, a composition that may be provided as a component in a patch, wherein the composition comprises an API-containing adhesive layer comprising, consisting essentially of, or consisting of a direct FXa which comprises or consists of one or more of betrixaban, edoxaban, fondaparinux, rivaroxaban, darexaban, otamixaban, letaxaban, eribaxaban and/or, desirably, apixaban, and preferably apixaban alone, a C₃-C₆ fatty acid, a glycol ether, and an adhesive.

In a related aspect, a patch may be provided by laminating the inventive composition onto a backing layer, and, optionally, covering the exposed composition with a release layer, the latter layer being removed prior to contacting the composition with a patient's skin.

It was unexpectedly found that the combination of a direct FXa which comprises or consists of one or more of betrixaban, edoxaban, fondaparinux, rivaroxaban, darexaban, otamixaban, letaxaban, eribaxaban and/or, desirably, apixaban, and preferably apixaban alone, with a C₃-C₆ fatty acid, a glycol ether, and adhesive, desirably certain fatty acids, glycol ethers and adhesives, and more desirably in certain amounts, provide an API-containing adhesive layer having desirable direct FXa drug release and/or flux values which are correlated with therapeutic concentrations of these direct FXas in the bloodstream.

The present invention also provides, in another aspect, a method for the preparation of a transdermal patch. The method comprises, consists essentially of, or consists of mixing a direct FXa which comprises or consists of one or more of betrixaban, edoxaban, fondaparinux, rivaroxaban, darexaban, otamixaban, letaxaban, eribaxaban and/or, desirably, apixaban, and preferably apixaban alone, a C₃-C₆ fatty acid and a glycol ether to provide a pre-mix, and adding an adhesive to the pre-mix followed by mixing, thereby providing a liquid; applying the liquid onto a release liner; drying the liquid to provide a coating on the release liner, and laminating the coating onto a backing layer.

In another aspect, the present invention provides methods for administering a direct FXa which comprises or consists of one or more of betrixaban, edoxaban, fondaparinux, rivaroxaban, darexaban, otamixaban, letaxaban, eribaxaban and/or, desirably, apixaban, and preferably apixaban alone, to a patient in need comprising applying a transdermal patch onto the skin of the patient, the patch comprising, consisting essentially of, or consisting of a backing layer and an API-containing adhesive layer comprising, consisting essentially of, or consisting of a direct FXa which comprises or consists of one or more of betrixaban, edoxaban, fondaparinux, rivaroxaban, darexaban, otamixaban, letaxaban, eribaxaban and/or, desirably, apixaban, and preferably apixaban alone, a C₃-C₆ fatty acid, a glycol ether, and adhesive, wherein the API-containing adhesive layer is in direct contact with the skin and, in preferred aspects, further provides desirable direct FXa drug release and/or flux values and/or FXa concentrations in the patient's bloodstream.

As used herein, “treatment” or “therapeutic benefit” includes relief or reduction of symptoms, as well as prophylaxis of symptoms, while a “therapeutically effective” amount refers to an amount of direct FXa in the API-containing adhesive layer that will provide a therapeutic benefit to a patient in need. Examples of conditions that may be treated using the inventive direct FXa transdermal patches are the same as those for which corresponding oral FXa formulations are approved, including as an anticoagulant in the treatment, including prevention, or reduction in the risk of blood clots (thromboembolism, e.g., deep vein thrombosis, pulmonary embolism) in the circulatory system, and in particular after (and in some cases also before) knee and hip replacement surgery, and to prevent or reduce the risk of stroke and embolism in people with atrial fibrillation (AF).

Thus, aspects of the present invention include the use of the API-containing adhesive layer, or the transdermal patch including that layer, in the treatment of a patient afflicted with thromboembolism, in the treatment of a patient afflicted with atrial fibrillation and is at risk for stroke and/or an embolism, and/or in the treatment of a patient that will undergo (e.g., within one week), or has undergone (e.g., within that past one week), a knee or hip replacement. The present invention is also provided in further aspects, including pharmaceutical compositions for the treatment of a patient applicated with a thromboembolism, a patient afflicted with atrial fibrillation and is at risk for stroke and/or an embolism, and/or a patient that will undergo within one week, or has undergone within one week, a knee or hip replacement.

The APIs useful in the various aspect of the present invention are well known, and comprise, consist essentially of, or consist of one or more of betrixaban, edoxaban, fondaparinux, rivaroxaban, darexaban, otamixaban, letaxaban, eribaxaban and, desirably, apixaban, and preferably apixaban alone. These APIs are poorly water-soluble: betrixaban (about 2.5-2.7 mg/ml), edoxaban (about 0.093 mg/mL), fondaparinux (about 7.91 mg/mL), rivaroxaban (about 0.01 mg/mL), darexaban (about 0.0048 mg/mL), otamixaban (about 0.0088 mg/mL), letaxaban (about 0.217 mg/mL), eribaxaban (about 0.00717 mg/mL) and apixaban (about 0.11 mg/ml). Apixaban is currently commercially available in the US only in tablet form for oral administration, and is a preferred API in the embodiments of the invention described herein.

Desirably, these APIs are substantially non-crystalline in the API-containing adhesive layer after it has been coated and dried, i.e., at least about 50%, 60%, 70%, 80%, 90% or 95% non-crystalline, as these crystalline APIs are relatively less soluble in the other adhesive layer formulation ingredients, resulting in a relatively lower flux. One means of reducing the crystallinity of these APIs in the coated and dried API-containing adhesive layer is to include a crystal inhibitor, such as, for example, crospovidone, therein. This inhibitor, when included, may be present in the API-containing adhesive layer in amounts ranging from about 5 wt. % to about 35 wt. %, desirably from about 7 wt. % to about 25 wt. %, more desirably from about 10 wt. % to about 20 wt. %, and even more desirably from about 12 wt. % to about 18 wt. %.

The amount of the aforementioned direct FXa APIs in the embodiments of the invention may vary, but generally may be included in the API-containing adhesive layer in an amount sufficient to provide the FXa concentration in the bloodstream which is approximately the same as that provided by daily dosing of oral formulations of the same FXa. For example, for AF patients, the administration of apixaban (based on data from the ELIQUIS® ARISTOTLE trial): (i) twice daily (2.5 mg each dose) provided apixaban steady state plasma concentrations (reached after 3 days of administration) ranging from about 90 to about 320 ng/ml (peak) and from about 40 to about 230 ng/ml (trough) and (ii) twice daily (5 mg each dose) provided apixaban steady state plasma concentrations (reached after 3 days of administration) ranging from about 70 to about 220 ng/mL (peak) and from about 30 to about 160 ng/ml (trough). By way of further example, for the treatment of venous thromboembolism, the administration of apixaban (based on data from the ELIQUIS® AMPLIFY trial): (i) twice daily (2.5 mg each dose) provided apixaban steady state plasma concentrations (reached after 3 days of administration) ranging from about 30 to about 160 ng/mL (peak) and from about 10 to about 100 ng/ml (trough), (ii) twice daily (5 mg each dose) provided apixaban steady state plasma concentrations (reached after 3 days of administration) ranging from about 60 to about 100 ng/ml (peak) and from about 20 to about 80 ng/mL (trough), and (iii) twice daily (10 mg each dose) provided apixaban steady state plasma concentrations (reached after 3 days of administration) ranging from about 100 to about 575 ng/ml (peak) and from about 40 to about 340 ng/ml (trough). All concentration ranges are provided as +/−2 SD. See also ELIQUIS® Package Insert (Revised: September 2021) accessed at https://packageinserts.bms.com/pi/pi_eliquis.pdf.

Generally, and on a weight percent basis, the amount of direct FXa included in the various embodiments may range from about 2 wt. % to about 20 wt. %, more desirably from about 3 wt. % to about 15 wt. %, even more desirably from about 4 wt. % to about 12 wt. %, more desirably from about 5 wt. % to about 10 wt. %, and even more desirably from about 6 wt. % to about 8 wt. %. In absolute amounts, the direct FXa APIs may be included in widely ranging amounts, as the amount is dependent upon the final (after drying) weight and thickness of the API-containing adhesive layer, the area of that layer (also referred to as patch size) that contacts the skin, and the desired dosing regimen. For example, it is contemplated that the inventive transdermal patches may be provided in varying doses, wherein the variation in dosing is proportional to the size of the API-containing adhesive layer that contacts the skin. Accordingly, and due to the different patch sizes and associated dosing variations, the absolute amount of direct FXa APIs in the API-containing adhesive layer of a particular patch may range from about 0.1 mg to about 400 mg, from about 0.5 mg to about 300 mg, or from about 1 to about 200 mg.

The length of administration, in combination with the dose, also may vary depending on the treatment. For reduction in the risk of stroke and embolism in patients with atrial fibrillation a dose should be administered that is equivalent to 5 mg taken orally twice daily, or 2.5 mg taken orally twice daily in patients with at least two of the following characteristics: age greater than or equal to 80 years; body weight less than or equal to 60 kg; serum creatinine greater than or equal to 1.5 mg/dL. For prophylaxis of thrombosis (e.g., deep vein thrombosis) following hip or knee replacement surgery, a dose should be administered that is equivalent to 2.5 mg taken orally twice daily, with an initial dose administered 12 to 24 hours after surgery, and, in patients undergoing hip replacement surgery for 35 days and in patients undergoing knee replacement surgery for 12 days. For the treatment of deep vein thrombosis and pulmonary embolism, a dose should be administered that is equivalent to 10 mg taken orally twice daily for the first 7 days, and 5 mg taken orally thereafter. For reduction in the risk of recurrence of deep vein thrombosis or pulmonary embolism, a dose should be administered that is equivalent to 2.5 mg taken orally twice daily after at least 6 months of treatment for deep vein thrombosis or pulmonary embolism.

Further, and to the extent it may be desirable to ensure maintenance of the concentration of the direct FXa API within a therapeutic range over an extended period of time, the administration of a direct FXa API in accordance with the present invention may be supplemented by the oral administration a direct FXa API, e.g., for one day (one or two doses) or optionally for two days, starting on the first day the API-containing adhesive layer is used on a patient or on any day a depleted API-containing adhesive layer is removed from a patient's skin (i.e., after the patient has worn the adhesive layer for an appropriate number of days) and is replaced by a new API-containing adhesive layer. The API-containing adhesive layer may be co-administered with an oral direct FXa API formulation, wherein the oral formulation is administered at a relatively low dose such that the combined administration of the direct FXa API by the oral formulation and API-containing adhesive layer are sufficient to provide the direct FXa API at desired therapeutic levels, while also providing a reduction in undesirable side effects experienced at relatively higher oral doses. The supplemental administration or co-administration may be provided to a patient in the form of a kit comprising one or more individual oral formulations with the API-containing adhesive layer.

In addition to the one or more direct FXa APIs, the API-containing adhesive layer in the various embodiments of the invention further include a C₃-C₆ fatty acid, and preferably lactic acid. The fatty acid may be included in the following amounts: from about 5 wt. % to about 35 wt. %, desirably about 10 wt. % to about 30 wt. %, more desirably from about 15 wt. % to about 25 wt. %, and more desirably from about 18 wt. % to about 22 wt. %.

It was further unexpectedly found that the inclusion of a glycol (or phenol) ether, and preferably 2-phenoxyethanol, in a relatively significant amount in the API-containing adhesive layer, contributed to, at least in part, a relatively high FXa flux. This ingredient may be present in the API-containing adhesive layer in the various embodiments of the invention from about 5 wt. % to about 35 wt. %, desirably about 10 wt. % to about 30 wt. %, more desirably from about 15 wt. % to about 25 wt. %, and even more desirably from about 18 wt. % to about 22 wt. %.

These ingredients are desirably also present in weight ratios (C₃-C₆ fatty acid: glycol ether) ranging from about 1:0.5 to about 1:2, more desirably from about 1:0.7 to about 1:1.5, even more desirably from about 1:0.8 to about 1:1.2, and most desirably a ratio of about 1:1.

The API-containing adhesive layer desirably further comprises a C₁₄-C₂₂ fatty acid, more desirably a C₁₆-C₂₀ fatty acid, preferably unsaturated, and most preferably linoleic acid. When included, this fatty acid is present in the adhesive layer at from about 1 wt. % to about 20 wt. %, desirably about 3 wt. % to about 15 wt. %, more desirably from about 4 wt. % to about 12 wt. %, and even more desirably from about 5 wt. % to about 10 wt. %.

These ingredients are desirably also present in weight ratios (C₃-C₆ fatty acid: C₁₄-C₂₂ fatty acid) ranging from about 0.5:1 to about 6:1, more desirably from about 0.7:1 to about 5:1, even more desirably from about 0.8:1 to about 4:1, and most desirably at a weight ratio of about 3:1.

When the adhesive layer includes a C₁₄-C₂₂ fatty acid, a C₃-C₆ fatty acid and a glycol ether, it was unexpectedly found that the ingredients in the adhesive layer are not only compatible, but the FXa flux is relatively high even when relatively lower levels of an FXa crystallinity inhibitor is included.

The API-containing adhesive layer desirably also comprises adhesives, and desirably a plurality of adhesives. More desirably, the plurality of adhesives comprise (meth) acrylic- and silicone-based adhesives. For purposes of the invention, (meth) acryl shall refer to both methacryl- as well as acryl-based adhesives.

Suitable acrylate-based adhesives include polyacrylate-based pressure sensitive adhesives (PSAs). These adhesives are well known, and include, by way of example and not limitation, adhesives marketed under the trademark DURO-TAK, including the 87 and 387 series, e.g., Duro-Tak 87-202A; Duro-Tak 87-208A; Duro-Tak 87-502A: Duro-Tak 87-503A; Duro-Tak 87-2051; Duro-Tak 87-2054; Duro-Tak 87-2287; Duro-Tak 87-2353; Duro-Tak 87-2510; Duro-Tak 87-2516; Duro-Tak 87-4098; Duro-Tak 87-4287; Duro-Tak 87-9088; and Duro-Tak 87-9301; and Duro-Tak 87-2516, adhesives marketed under the trademark GELVAR, including, e.g., Gelva GMS 9083, Gelva GMS 9073, Gelva GMS 3083, Gelva GMS 788, Gelva GMS3253, Gelva GMS 737. Gelva GMS 3087, Gelva GMS 3235, all available from Henkel Corporation. Duro-Tak 87-4098 is preferred, as it provides the adhesive layer with the desired hardness and/or assists in obtaining the desired viscosity.

Suitable silicone-based PSAs also are well known, and include, but are not limited to, adhesives marketed by DuPont under the Liveo™ Bio-PSA trademark, e.g., BIO-PSA 7-4101, BIO-PSA 7-4103, BIO-PSA 7-4201, BIO-PSA 7-4203, BIO-PSA 7-4301, BIO-PSA 7-4303, BIO-PSA 7-4102, BIO-PSA 7-4202, BIO-PSA 7-4302, BIO-PSA 7-4401, BIO-PSA 7-4403, BIO-PSA 7-4501, BIO-PSA 7-4503, BIO-PSA 7-4601, BIO-PSA 7-4603, BIO-PSA 7-4402, BIO-PSA 7-4502, BIO-PSA 7-4602, BIO-PSA 7-4560, BIO-PSA 7-4402, BIO-PSA 7-4408 Cosmetic Fluid, 7-4405 Cosmetic Fluid, 7-4408 Cosmetic Fluid, 7-4411 Cosmetic Fluid, 7-4412 Cosmetic Fluid, and 7-4413 Cosmetic Fluid. Uncapped polymers are preferred, e.g., BIO-PSA 7-4402 (a polydimethyl siloxane polymer), as such polymers contribute to providing the adhesive layer with the desired hardness, skin adhesion and/or drug permeability. Skin adhesion may vary based upon the number of silanol functional groups available for hydrogen bonding, with a greater number of functional groups generally expected to correlate with increased adhesion. It is known to those skilled in the art that silicone-based adhesives provide higher drug permeability rates relative to those provided when using other classes of adhesive, e.g., acrylate-based adhesives. Thus, the drug diffusion rate for drug-containing compositions may be adjusted, in part, by modifying the weight ratio of silicone-based adhesive to non-silicone-based adhesive.

The amount of adhesive to be included in the API-containing adhesive layer may generally range from about 15 wt. % to about 90 wt. %, desirably from about 25 wt. % to about 65 wt. %, more desirably from about 35 wt. % to about 55 wt. %, and even more desirably from about 40 wt. % to about 50 wt. %. When both acrylate-and silicone-based adhesives are included, as preferred, they desirably may be present (acrylate: silicone) in a weight ratio of about 1:0.7; more desirably about 1:0.9 and even more desirably about 1:1.

The API-containing adhesive layer may further, and optionally, include at least one of an antioxidant, a chelating agent, or both. While not desiring to be bound to any particular theory, the antioxidant may assist in stabilizing the API-containing adhesive layer by assisting in reducing, and preferably eliminating, any color change (e.g., yellowing) during the shelf life of the product and/or oxidation and associated degradation of the permeation enhancers, while the chelating agent may bind, or form a complex with, any trace metals that may be present in the ingredients present in the API-containing adhesive layer, thereby reducing, and desirably precluding, any ability of these metals to catalyze oxidation reactions.

Antioxidants that may be used in the present invention include, without limitation, alpha tocopherol, ascorbic acid, ascorbyl palmitate, butylated hydroxyanisole (BHA), butylated hydroxytoluene (BHT), monothioglycerol, potassium metabisulfite, propionic acid, propyl gallate, sodium ascorbate, sodium bisulfite, sodium metabisulfite, and sodium sulfite, with butylated hydroxytoluene being preferred. Chelating agents that may be used in the present invention include, without limitation, ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof.

If included, an antioxidant and chelating agent may each be present (alone or together) in an amount ranging from about 0.1 wt. % to about 2 wt. %, and more desirably from about 0.2 wt. % to about 1 wt. %, and even more desirably from about 0.4 wt. % to about 0.6 wt. %.

The weight percents of the ingredients in the API-containing adhesive layers described herein should be understood to be based on the total weight of the API-containing adhesive layer after drying, wherein drying is conducted in a manner that removes all volatile components from the API-containing adhesive layer. For example, the adhesives described herein are provided from the manufacturer as solids in a volatile liquid vehicle. The weight percent of an adhesive in an API-containing adhesive layer, as described herein, refers to the weight of the adhesive solids (i.e., excluding the volatile liquid vehicle) remaining in the API-containing adhesive layer after drying.

Table A provides a series of illustrative API-containing adhesive layer formulations in accordance with the present invention.

TABLE A

| | Formulation 1 | Formulation 2 | Formulation 3 | Formulation 4 |
|---|---|---|---|---|
| Ingredient | (wt. %) | (wt. %) | (wt. %) | (wt. %) |
| FXa | 2-20 | 3-15 | 4-12 | 5-8 |
| C₃-C₆ fatty acid | 5-35 | 10-30 | 15-25 | 18-22 |
| glycol ether | 5-35 | 10-30 | 15-25 | 18-22 |
| Acrylate-based | 5-45 | 10-35 | 15-30 | 20-25 |
| adhesive | | | | |
| Silicone-based | 5-45 | 10-35 | 15-30 | 20-25 |
| adhesive | | | | |
| C₁₆-C₂₀ fatty | 1-20 | 3-15 | 4-12 | 5-10 |
| acid (optional) | | | | |
| Crospovidone | 5-35 | 7-25 | 10-20 | 10-20 |
| (optional) | | | | |

The foregoing API-containing adhesive layer formulations may provide transdermal products that exhibit desirable drug release rates, as well as desirable flux values. Drug release rates of the API-containing adhesive layer, and desirably in embodiments wherein apixaban is the API, may range from at least about 30, 35 or 40% to about 45, 50, 55 or 60% of the drug from the adhesive layer over 1 day, and/or at least about 70, 75, 80, 85 or 90% and up to about 92, 95, 97 or 99% over 7 days, as determined by the methodology described in paragraphs-herein. Flux values of the API-containing adhesive layer, and desirably in embodiments wherein apixaban is the API, may range from at least about 2, 2.5, 3, 3.5, 4, 4.5, 5, 5.5, 6 or 6.5 and up to about 7, 7.5, 8, 9 or 10 μg/cm²/hr, over 1, 2, 3, 4, 5, 6 or 7 days, as determined by the methodology described in paragraphs-herein.

Embodiments of the invention provide a transdermal patch that can be worn for extended periods of time, such as for 3, 4, 5, 6, 7, 8 or 9 days while transdermally delivering therapeutically effective amounts of the aforesaid direct FXa APIs to a patient. The patch also may be used in place of an oral FXa should undesirable side effects arise in a patient ingesting the FXa orally, or after 1, 2 or 3 days of administration of an oral dosage form (e.g., to reduce any anticipated side effects associated with oral administration).

The API-containing adhesive layer, after coating and drying, may be laminated onto a backing layer that is impervious to the transmission of ingredients in the adhesive layer, with the exposed adhesive layer being covered by a release layer which is removed prior to use. The backing layer may be oversized relative to the area of the API-containing adhesive layer, with the area not covered by the adhesive layer having an adhesive coated thereon which may assist in retention of the API-containing adhesive layer on the skin. These backing layers are well known, and include, by way of illustration only, polyester films. The adhesive layer as described herein is designed to directly contact the skin, and as such no membrane covering the coated and dried adhesive layer is required.

### EXAMPLE 1

Illustrative transdermal apixaban formulations, as described in Tables 1 and 2, were prepared and tested to assess their crystallinity, dissolution and flux.

TABLE 1
Formulations (wt. %)

| Ingredients | | A | B | C | D | E | F | G |
|---|---|---|---|---|---|---|---|---|
| API | Apixaban | 10 | 10 | 10 | 15 | 6 | 6 | 6 |
| Enhancers | Lactic Acid | 0 | | | | | | |
| | Linoleic Acid | 0 | | | | | | |
| | 2- | 0 | | | | | | |
| | Phenoxyethanol | | | | | | | |
| | 1-methy1-2- | 0 | 5 | 10 | 0 | | | |
| | pyrrolidinone | | | | | | | |
| Si PSA | BioPSA 7-4202 | 0 | | | | | | |
| | BioPSA 7-4402 | 0 | | | | | | |
| | BioPSA 7-4502 | 0 | | | | | | |
| Acrylic | Duro-Tak | 0 | | | | | | |
| PSA | 87-4098 | | | | | | | |
| Acrylic | Gelva GMS 788 | 0 | | | | | | 94 |
| PSA | | | | | | | | |
| Acrylic | Duro-Tak 2516 | 0 | | | | | | |
| PSA | | | | | | | | |
| Acrylic | Duro-Tak 2287 | 0 | | | | | | |
| PSA | | | | | | | | |
| Acrylic | Gelva GMS 9083 | 0 | | | | | | |
| PSA | | | | | | | | |
| Acrylic | Duro-Tak 2051 | | | | | | 94 | |
| PSA | | | | | | | | |
| Acrylic | AS-223 | 90 | 85 | 80 | 85 | 94 | 0 | |
| PSA | | | | | | | | |
| Crystal | Crospovidone | 0 | | | | | | |
| inhibitor | (CL-M) | | | | | | | |
| Coatweight (mg/cm²) | | 6 | 6 | 6 | 6 | 6 | 6 | 6 |
| Testing | Crystals (Y/N) | N-I | N-1 | N-I | N-I | N-PS | N-PS | N-I |
| | Dissolution/ | 61.6% * | 57.5% * | 61.5% * | 62% * | 21.30% | 8.40% | N/A |
| | Drug Release % | | | | | | | |
| | @ 7 days | | | | | | | |
| | Flux_{0-7 days} | 0.43 ** | 0.25 ** | 0.63 | 0.15 ** | 0 | 0 | N/A |
| | (μg/hr/cm²) | | | | | | | |

| Ingredients | | H | I | J | K | L | M |
|---|---|---|---|---|---|---|---|
| API | Apixaban | 6 | 6 | 6 | 10 | 6 | 6 |
| Enhancers | Lactic Acid | 0 | | | | 10 | 20 |
| | Linoleic Acid | 0 | | | | | |
| | 2- | 0 | | | | 10 | 20 |
| | Phenoxyethanol | | | | | | |
| | 1-methy1-2- | 0 | | | | | |
| | pyrrolidinone | | | | | | |
| Si PSA | BioPSA 7-4202 | 0 | | | | | |
| | BioPSA 7-4402 | 0 | | | | | |
| | BioPSA 7-4502 | 0 | | | | | |
| Acrylic | Duro-Tak | 0 | | | 90 | 74 | 54 |
| PSA | 87-4098 | | | | | | |
| Acrylic | Gelva GMS 788 | 0 | | | | | |
| PSA | | | | | | | |
| Acrylic | Duro-Tak 2516 | 94 | 0 | | | | |
| PSA | | | | | | | |
| Acrylic | Duro-Tak 2287 | 0 | 94 | 0 | | | |
| PSA | | | | | | | |
| Acrylic | Gelva GMS 9083 | 0 | | 94 | 0 | | |
| PSA | | | | | | | |
| Acrylic | Duro-Tak 2051 | | | | | | |
| PSA | | | | | | | |
| Acrylic | AS-223 | 0 | | | | | |
| PSA | | | | | | | |
| Crystal | Crospovidone | 0 | | | | | |
| inhibitor | (CL-M) | | | | | | |
| Coatweight (mg/cm²) | | 6 | 6 | 6 | 6 | 15 | 15 |
| Testing | Crystals (Y/N) | N | N | N | N-I | N | N |
| | Dissolution/ | 3.70% | 4.70% | 10.50% | 7.9% * | 0.30% | 10.05% |
| | Drug Release % | | | | | | |
| | @ 7 days | | | | | | |
| | Flux_{0-7 days} | 0 | 0 | 0 | 0.08 ** | 0.19 | 0.71 |
| | (μg/hr/cm²) | | | | | | |

Key:
N = No crystals observed in coated layer up to 3 months after coating.
N-I = No crystals observed in coated layer up to 3 months after coating; all apixaban was not dissolved in formulation.
N-PS = No crystals observed in coated layer up to 3 months after coating; appearance of at least one area of the coated layer was
different relative to appearance of another area.
* Dissolution/Drug Release % @ 1 day
** Flux_{48-72 h} (μg/hr/cm²)

TABLE 2
Formulations (wt. %)

| Ingredients | | N | O | P | Q | R | S | T | U | V |
|---|---|---|---|---|---|---|---|---|---|---|
| API | Apixaban | 10 | 6 | 6 | 10 | 12.5 | 15 | 6 | 8 | 10 |
| Enhancers | Lactic Acid | 0 | 10 | 20 | 20 | 20 | 20 | 20 | 20 | 20 |
| | Linoleic Acid | | | | | | | | | |
| | 2-Phenoxy | 0 | 10 | 20 | 20 | 20 | 20 | 20 | 20 | 20 |
| | ethanol | | | | | | | | | |
| | 1-methy1-2- | | | | | | | | | |
| | pyrrolidinone | | | | | | | | | |
| Si PSA | BioPSA 7-4202 | 90 | 37 | 27 | 0 | | | | | |
| | BioPSA 7-4402 | | | | 25 | 23.75 | 22.5 | 22 | 21 | 20 |
| | BioPSA 7-4502 | | | | | | | | | |
| Acrylic | Duro-Tak | 0 | 37 | 27 | 25 | 23.75 | 22.5 | 22 | 21 | 20 |
| PSA | 87-4098 | | | | | | | | | |
| Crystal | Crospovidone | | | | | | | 10 | 10 | 10 |
| inhibitor | (CL-M) | | | | | | | | | |
| Coat weight (mg/cm²) | | 6 | 15 | 15 | 30 | 30 | N/A | 15 | 15 | 15 |
| Testing | Crystals (Y/N) | N-I | N | Y | Y | Y | Too | N | N | N |
| | Dissolution/ | 49.10%* | 15.09% | 27.43% | 14.67% | 19.87% | much | 91.64% | 83.23% | 80.09% |
| | Drug Release % | | | | | | API. | | | |
| | @ 7 days | | | | | | | | | |

| | | | | | | | |
|---|---|---|---|---|---|---|---|
| Flux_{0-7 days} | 1.53 ** | 0.74 | 1.51 | Not Tested | 3.36 | 5.5 | 5.53 |
| (μg/hr/cm²) | | | | | | | |

| Ingredients | | W | X | Y | Z | AA | BB | CC | DD | EE | FF |
|---|---|---|---|---|---|---|---|---|---|---|---|
| API | Apixaban | 12 | 6 | 8 | 10 | 12 | 6 | 6 | 6 | 6 | 6 |
| Enhancers | Lactic Acid | 20 | 20 | 20 | 20 | 20 | 20 | 20 | 20 | 0 | 15 |
| | Linoleic Acid | | | | | | | | | 20 | 5 |
| | 2-Phenoxy | 20 | 20 | 20 | 20 | 20 | 20 | 20 | 20 | 20 | 20 |
| | ethanol | | | | | | | | | | |
| | 1-methy1-2- | | | | | | | | | | |
| | pyrrolidinone | | | | | | | | | | |
| Si PSA | BioPSA 7-4202 | 0 | | | | | | | | | |
| | BioPSA 7-4402 | 19 | 0 | | | | 24.5 | 22 | 19.5 | 22 | 22 |
| | BioPSA 7-4502 | | 22 | 21 | 20 | 19 | 0 | | | | |
| Acrylic | Duro-Tak | 19 | 22 | 21 | 20 | 19 | 24.5 | 22 | 19.5 | 22 | 22 |
| PSA | 87-4098 | | | | | | | | | | |
| Crystal | Crospovidone | 10 | 10 | 10 | 10 | 10 | 5 | 10 | 15 | 10 | 10 |
| inhibitor | (CL-M) | | | | | | | | | | |

| | | | | | | | | | | |
|---|---|---|---|---|---|---|---|---|---|---|
| Coat weight (mg/cm²) | 15 | 30 | 30 | 30 | 30 | 30 | 30 | 30 | N/A | 30 |

| | | | | | | | | | |
|---|---|---|---|---|---|---|---|---|---|
| Testing | Crystals (Y/N) | N | N | N | N | N | In | N-I | In |
| | | | | | | | observation | | observation |

| Dissolution/ | 84.08% | Not Tested | | | | | | | Not Tested | |
|---|---|---|---|---|---|---|---|---|---|---|
| Drug Release % | | | | | | | | | | |
| @ 7 days | | | | | | | | | | |
| Flux_{0-7 days} | 5.14 | 4.23 | 3.76 | 3.05 | 3.48 | 1.69 | 5.41 | 6.48 | | 6.66 |
| (μg/hr/cm²) | | | | | | | | | | |

Key:
N = No crystals observed in coated layer up to 3 months after coating.
N-I = No crystals observed in coated layer up to 3 months after coating; all apixaban was not dissolved in formulation.
*Dissolution/Drug Release % @ 1 day
** Flux_{48-72 h} (μg/hr/cm²)

The formulations of Tables 1 and 2 were prepared as follows. The apixaban was introduced into a 100 ml bottle. Lactic acid, linoleic acid, 2-phenoxyethanol and/or 1-methyl-2-pyrrolidinone were then added thereto. The resulting suspension was vortexed, and the bottle placed on a vial rocker until the apixaban was completely dissolved. The crospovidone, if included, was then added to the solution, and mixed for 2 minutes (@2000 rpm). The silicone-based pressure sensitive adhesive (SiPSA) and/or acrylic-based PSA were then added, and the resulting mixture was mixed for 4 minutes (@2000 rpm). The resulting formulation was an opaque, white-colored, viscous liquid.

Thereafter, the liquid formulation was coated onto a 3 mil Scotchpak™ 9709 fluorosilicone release liner (a fluorosilicone-coated polyester film) using a manual knife-over-roll lab coater with a gap of 0.017 inch. The coated formulation was then dried for 10 min at room temperature, and then for 10 min at 70° C. After drying, the formulation was laminated onto a 2 mil polyester backing film to provide a transdermal dosage form. The actual weights of the dry coating layers ranged between 6 mg/cm² to 30 mg/cm², as listed in the Tables.

The dissolution/drug release values reported in this example were obtained as follows. A jacketed Franz cell with 8 mL receiver volume and 1.00 cm² receiver chamber orifice area was used. The receiver media was 32.5° C. 1% Sodium Dodecyl Sulfate (SDS) in Dulbecco's Phosphate Buffered Saline (DPBS) without CaCl₂ and MgCl₂.

Samples of the laminated transdermal product were cut to 1.6 cm². The release liner on the samples was then removed, and the sample product was placed onto the Franz receiver cell, completely covering the 1.0 cm² orifice area. The donor chamber was then affixed onto the receiver cell, thereby completing assembly of the test unit. 8.0 ml (preheated to 32.5° C.) of the receiver media was introduced into the receiver chamber, and a timer was started. Sampling of the receiver media was undertaken at the following time points: 6, 24, 48, 78, 143 and 168 hours (unless otherwise indicated in the tables). After sampling at each time interval, the sampled receiver media from the Franz cell was transferred to individual HPLC vials and assayed for FXa concentration using a HPLC method. After each sampling was completed, the remaining contents in the receiver chamber were removed, and 8.0 ml of fresh preheated (to 32.5° C.) receiver media was introduced into the receiver chamber. The drug release was then calculated for each sample.

Reference in Tables 1 and 2 to “Crystals (Y/N)” refers to a visual observation of the adhesive layer after casting. A “Y” indicates that FXa crystals (in this case apixaban crystals) were observed by compound microscope (at up to 400× magnification) or by the naked eye, e.g., as needle-or fan-shaped objects, the presence of such crystal being undesirable. A “N' indicates that no such crystals were observed by either the naked eye or at 400×. These Y/N values reflect observations of the adhesive layers undertaken through 3 months post-casting.

The data in Table 2 shows that crystal formation was not present in formulations comprising crospovidone, wherein those formulations included a 1:1 weight ratio of 7-4402 silicone-based adhesive: 87-4098 acrylic-based adhesive. Complete (i.e., 100%) dissolution/drug release was achieved only for formulations that included crospovidone. Further, the inclusion of 1-methyl-2-pyrrolidinone, a solvent for apixaban, while apparently assisting in providing the formulation with a reasonable drug release, did not assist in providing the formulation with a desirable apixaban flux.

### EXAMPLE 2

An illustrative transdermal apixaban formulation, as described in Table 3, was prepared and tested as described in Example 1 to obtain its apixaban drug release profile.

TABLE 3

| Ingredient | wt. % |
|---|---|
| Apixaban | 6 |
| Lactic Acid | 20 |
| 2-Phenoxyethanol | 20 |
| BioPSA 7-4402 | 22 |
| Duro-Tak 87-4098 | 22 |
| Crospovidone (CL-M) | 10 |

The testing of this formulation was conducted in triplicate, with the average drug release values of the three runs being shown in FIG. 1.

The example demonstrates that about 100% of the drug is released within 7 days (168 hours).

### EXAMPLE 3

Illustrative transdermal apixaban formulations as described in Table 4 were prepared and tested over a 3-or 7-day period to obtain their skin permeation (flux) value, which values were also compared relative to a known transdermal apixaban formulation.

The formulations of Examples 2, 3 and 4 were prepared as described in Example 1. A known apixaban formulation, Example 22 from US Published Patent Application No. US20200338012A1, was prepared as follows: 1.71 g of apixaban was weighed into a 100 ml glass jar, and then 9.40 g of acrylic acid, 9.40 g of lactic acid, 6.41 g of octanoic acid, 1.71 g of Eugenol and 4.28 g of methyl lactate were weighed and introduced into the jar. The jar contents then were vortexed until the apixaban was completely dissolved. 42.21 g of Duro-Tak 87-2510 (40.5% solid) then was added to the apixaban solution and mixed for 4 min@2,000 rpm. Thereafter, each liquid formulation was coated onto a 3 mil Scotchpak™ 9709 fluorosilicone release liner using a manual knife-over-roll lab coater with a gap of 0.017 inch. The coated formulation was then dried for 10 min at room temperature, and then for 10 min at 70° C. After drying, the formulation was laminated onto a 2 mil polyester backing film to provide a transdermal dosage form. The actual weight of the dry coating layer was 6 mg/cm².

The in vitro cadaver skin permeation profiles (flux values) reported in this example were obtained using the following methodology. A jacketed Franz cell with 8 mL receiver volume and 1.00 cm² receiver chamber orifice area was used. The receiver media was 32.5° C. 1% Sodium Dodecyl Sulfate (SDS) in Dulbecco's Phosphate Buffered Saline (DPBS) without CaCl₂ and MgCl₂.

Frozen donor cadaver skin from the posterior torso was received and utilized for these tests. The cadaver skin was rapidly thawed in 37.5° C. DPBS for 20 minutes. 6.24 cm² round-shaped skin specimens were die-cut from the thawed cadaver skin and immediately placed over the receiver chamber of each vertical Franz cell with the stratum corneum side facing up. 32.5° C. ˜6 ml receiver media was transferred into each jacketed Franz cell and the cadaver skin was allowed to equilibrate at 32.5° C. for one hour with magnetic stirring.

The testing thereafter commenced by die-cutting 0.785 cm² samples from each transdermal dosage sample to be tested. The release liner on the samples was then removed, and the sample was placed onto the center of the Franz receiver cell comprising the cadaver skin. PET film was die-cut (in a round shape—at 3.89 cm²) and placed over the test sample to assist in ensuring complete contact between the test sample and the skin. The donor chamber was then affixed onto the receiver cell, thereby completing assembly of the test unit. 8.0 ml (preheated to 32.5° C.) of the receiver media was introduced into the receiver chamber, and a timer was started. Sampling of the receiver media was undertaken at the following time points: 6, 24, 48, 78, 143 and 168 hours (with the exception of Example 22 from US Published Patent Application No. US20200338012A1, wherein sampling was halted at 72 hours). After sampling at each time interval, the sampled receiver media from the Franz cell was transferred to individual HPLC vials and assayed for apixaban concentration using an HPLC method. After each sampling was completed, the remaining contents in the receiver chamber were removed, and 8.0 ml of fresh preheated 32.5° C. receiver media was introduced into the receiver chamber. The following were then calculated for each sample: cumulative delivery: μg/cm²/7 day; % apixaban delivered after 7 days; Flux₀₋₁₆₈ₕᵣₛ: μg/cm²/hr; and t_{lag}: hours. Values obtained for the formulation described in Example 22 from US Published Patent Application No. US20200338012A1, at different time points, also were calculated.

The testing of each formulation was conducted in triplicate, with the average values of the three runs being reported in Table 4.

TABLE 4

| | Formulation | Formulation | Formulation | Example #22 from |
|---|---|---|---|---|
| | Example 2 | Example 3 | Example 4 | US20200338012A1 |
| Ingredient | (wt. %) | (wt. %) | (wt. %) | (wt. %) |
| Apixaban | 6 | 6 | 6 | 3.42 |
| Lactic Acid | 20 | 20 | 15 | 18.8 |
| Linoleic Acid | 0 | 0 | 5 | 0 |
| 2-Phenoxyethanol | 20 | 20 | 20 | 0 |
| BioPSA 7-4402 | 22 | 19.5 | 22 | 0 |
| Acrylic Acid | 0 | 0 | 0 | 18.8 |
| Octanoic Acid | 0 | 0 | 0 | 12.82 |
| Eugenol | 0 | 0 | 0 | 3.42 |
| Methyl-1-Lactate | 0 | 0 | 0 | 8.55 |
| Duro-Tak 87-2510 | 0 | 0 | 0 | 34.19 |
| Duro-Tak 87-4098 | 22 | 19.5 | 22 | 0 |
| Crospovidone (CL-M) | 10 | 15 | 10 | 0 |
| Flux (μg/cm²/hr) | 3.36 | 6.48 | 6.66 | 0.18 |
| | (0-168 hours) | (0-168 hours) | (0-168 hours) | (0-72 hours) |

Certain results are reported graphically in the following figures.

FIG. 2 provides the average (over 3 runs) cumulative apixaban delivery profile for each formulation in Table 4.

FIG. 3 provides the average (over 3 runs) apixaban flux profile for each formulation in Table 4 at various time intervals.

The data indicate that Formulation Examples 2, 3 and 4 provided significantly higher drug deliveries and flux profiles relative to Example 22 of US Published Patent Application No. US20200338012A1.

All references, including publications, patent applications, and patents, cited herein are hereby incorporated by reference to the same extent as if each reference were individually and specifically indicated to be incorporated by reference and were set forth in its entirety herein.

References to weight percents herein should be understood to describe an amount of a component or ingredient based on the total weight of the adhesive layer formulation absent any volatiles which are removed upon drying (the “dry composition”), unless contradicted by express language or context.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “illustrative,” “such as”) provided herein, is intended merely to better illuminate the invention and does not pose a limitation on the scope of the invention unless otherwise claimed. No language in the specification should be construed as indicating any non-claimed element as essential to the practice of the invention.

Although preferred embodiments of this invention are described herein, variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1.-55. (canceled)

56. A formulation comprising:
(a) about 2 wt. % to about 20 wt. % apixaban;
(b) about 5 wt. % to about 35 wt. % lactic acid;
(c) about 5 wt. % to about 35 wt. % of a glycol ether;
(d) about 5 wt. % to about 45 wt. % of an acrylate-based adhesive; and
(e) about 5 wt. % to about 45 wt. % of a silicone adhesive,
wherein the weight percents are based on the total weight of the formulation after removal of any volatiles therefrom.

57. The formulation of claim 56, further comprising crospovidone.

58. The formulation of claim 57, wherein the crospovidone is present in the formulation in an amount ranging from about 5 wt. % to about 35 wt. %, wherein the weight percent is based on the total weight of the formulation after removal of any volatiles therefrom.

59. The formulation of claim 57, further comprising a C₁₆-C₂₀ fatty acid.

60. The formulation of claim 59, wherein the C₁₆-C₂₀ fatty acid is present in the formulation in an amount ranging from about 1 wt. % to about 20 wt. %, wherein the weight percent is based on the total weight of the formulation after removal of any volatiles therefrom.

61. The formulation of claim 56, wherein the glycol ether is 2-phenoxyethanol.

62. The formulation of claim 57, wherein apixaban is present in the formulation in an amount ranging from about 4 wt. % to about 12 wt. %; lactic acid is present in the formulation in an amount ranging from about 15 wt. % to about 25 wt. %; the glycol ether is 2-phenoxyethanol and is present in the formulation in an amount ranging from about 15 wt. % to about 25 wt. %; the acrylate-based adhesive is present in the formulation in an amount ranging from about 15 wt. % to about 30 wt. %; the silicone-based adhesive is present in the formulation in an amount ranging from about 15 wt. % to about 30 wt. %; and crospovidone is present in the formulation in an amount ranging from about 10 wt. % to about 20 wt. %.

63. The formulation of claim 57, wherein apixaban is present in the formulation in an amount ranging from about 5 wt. % to about 8 wt. %; lactic acid is present in the formulation in an amount ranging from about 18 wt. % to about 22 wt. %; the glycol ether is 2-phenoxyethanol and is present in the formulation in an amount ranging from about 18 wt. % to about 22 wt. %; the acrylate-based adhesive is present in the formulation in an amount ranging from about 20 wt. % to about 25 wt. %; the silicone-based adhesive is present in the formulation in an amount ranging from about 20 wt. % to about 25 wt. %; and crospovidone is present in the formulation in an amount ranging from about 10 wt. % to about 20 wt. %.

64. The formulation of claim 56, further comprising an antioxidant, a chelating agent, or a combination thereof.

65. A formulation comprising:
(a) about 6 wt. % apixaban:
(b) about 15 wt. % to about 20 wt. % lactic acid;
(c) about 20 wt. % 2-phenoxyethanol ether;
(d) about 10 wt. % to about 15 wt. % crospovidone:
(e) an acrylate-based adhesive: and
(f) a silicone adhesive,
wherein the weight percents are based on the total weight of the formulation after removal of any volatiles therefrom.

66. The formulation of claim 65, further comprising about 0 wt. % to about 5 wt. % linoleic acid.

67. The formulation of claim 65, wherein the acrylate-based and silicone-based adhesives are present in the formulation at a weight ratio of about 1:1.

68. A transdermal patch comprising the formulation of claim 56 and a backing layer.

69. A transdermal patch comprising the formulation of claim 65 and a backing layer.

70. A method for transdermally administering apixaban to a patient in need comprising
providing a formulation comprising:
(a) about 2 wt. % to about 20 wt. % apixaban;
(b) about 5 wt. % to about 35 wt. % lactic acid;
(c) about 5 wt. % to about 35 wt. % of a glycol ether;
(d) about 5 wt. % to about 45 wt. % of an acrylate-based adhesive; and
(e) about 5 wt. % to about 45 wt. % of a silicone adhesive,
wherein the weight percents are based on the total weight of the formulation after removal of any volatiles therefrom, and
applying the formulation onto the skin of the patient, wherein the formulation contacts the skin.

71. The method of claim 70, wherein the patient in need is afflicted with a thromboembolism.

72. The method of claim 70, wherein the patient in need will undergo, within one week, a knee or hip replacement.

73. The method of claim 70, wherein the patient in need is afflicted with atrial fibrillation and is at risk for stroke and/or an embolism.

74. The method of claim 70, wherein the patient in need has undergone a knee or hip replacement.

75. The method of claim 70, wherein the patient in need was previously afflicted with deep vein thrombosis or pulmonary embolism.

76. The method of claim 70, further comprising administering an oral dosage form comprising apixaban to the patient in need, wherein the combined administration of the apixaban transdermally and orally provides the patient with a concentration of apixaban in the patient's blood at desired therapeutic levels.
